(19) United States
(12) Patent Application Publication
(10) Pub. No.: US 2019/0091118 A1
(43) Pub. Date: Mar. 28, 2019

(54) AGENT FOR TREATING DYED HAIR

(71) Applicant: Henkel AG & Co. KGaA, Dusesseldorf (DE)

(72) Inventors: Erik Schulze zur Wiesche, Hamburg (DE); René Krohn, Norderstedt (DE)

(73) Assignee: Henkel AG & Co. KGaA, Duesseldorf (DE)

(21) Appl. No.: 15/536,657

(22) PCT Filed: Dec. 3, 2015
(86) PCT No.: PCT/EP2015/078452

§ 371 Date: Jun. 16, 2017

(30) Foreign Application Priority Data
Dec. 17, 2014 (DE) 102014226176.7

(51) Int. Cl. A61K 8/26; A61Q 5/02; A61Q 5/12

(52) U.S. Cl. CPC A61K 8/26; A61Q 5/004; A61Q 5/12; A61Q 5/02

(57) ABSTRACT

A composition for treating keratinous fibers is provided herein. The composition includes, relative to the weight thereof, from about 0.01 to about 4.00 wt % of at least one alum. The composition further includes, relative to the weight thereof, from about 0.01 to about 5.00 wt % of at least one salt which comprises a bivalent cation.

### CROSS-REFERENCE TO RELATED APPLICATION

This application is a U.S. National-Stage entry under 35 U.S.C. § 371 based on International Application no. PCT/EP2015/078452 filed Dec. 3, 2015, which was published under PCT Article 21(2) and which claims priority to Application no. 10 2014 226 176.7 filed Dec. 17, 2014, which are all hereby incorporated in their entirety by reference.

### TECHNICAL FIELD

The invention is situated in the cosmetics domain and pertains to compositions for treating keratinous fibers which include at least one alum and at least one salt that contains a bivalent cation. The invention further relates to a method for preserving the color of dyed keratinous fibers and the use of at least one alum and at least one salts that contains a bivalent cation in cosmetic hair treatment compositions for preserving the color of dyed hair.

Products for altering hair color figure prominently in hair cosmetics. A distinction is made between permanent, semipermanent and temporary coloring systems, and these are based on chemical and/or natural dyes. However, a disadvantage of the hair colors which are created artificially using permanent, semipermanent or temporary coloring systems is that they can change in a way that is unwelcome—for example when the hair is washed. The phrase “unwelcome change” is understood to mean bleaching or bleeding as well as the loss of the luster of the shade of hair color achieved with the respective dye. Environmental influences and/or the effects of the Sun can make these changes more pronounced still. Accordingly, there is a need for cosmetic compositions with which artificially created hair colors can be stabilized more effectively.

### BACKGROUND

Hair treatment compositions for protecting artificially created hair colors are known in principle. Such hair treatment compositions are typically based on silicones, polymers or certain UV filter substances. EP 1676604 A1 describes a multistage method for improving the shade of hair color in which the hair is washed in one step with a shampoo which among other ingredients contains a water-soluble salt, preferably sodium sulphate.

However, the known compounds cannot adequately address all of the requirements placed on color preservers. Particularly the coloring of fragile, very fine and/or damaged hair is difficult and must not be repeated too often to avoid stressing the hair further. In these cases, it is particularly worthwhile to attempt to keep the created hair color unchanged as far as possible for a long period by treating the hair with suitable agents. In addition, the hair treatment compositions should optimally offer a conditioning benefit as well particularly for fragile, very fine and/or damaged hair.

### BRIEF SUMMARY

A composition for treating keratinous fibers is provided herein. The composition includes, relative to the weight thereof, from about 0.01 to about 4.00 wt % of at least one alum. The composition further includes, relative to the weight thereof, from about 0.01 to about 5.00 wt % of at least one salt which comprises a bivalent cation.

Another composition for treating keratinous fibers is provided herein. The composition includes, relative to the weight thereof, from about 0.05 to about 3.50 wt % of at least one alum of formula MIAl(SO₄)₂×12H₂O, in which MI stands for a sodium ion, a potassium ion, an ammonium ion, or a guanidinium ion. The composition further includes, relative to the weight thereof, from about 0.02 to about 4.50 wt % of calcium lactate and/or magnesium lactate. The composition also includes, relative to the weight thereof, from about 2.00 to about 18.00 wt % of at least one anionic surfactant and/or from about 0.15 to about 8.00 wt % of at least one cationic surfactant. The composition further includes, relative to the weight thereof, from about 0.01 to about 3.00 wt % of at least one of the cationic polymers known by the INCI designators Polyquaternium-10, Guar Hydroxypropyltrimonium Chloride, Polyquaternium-37, Polyquaternium-67 and/or Polyquaternium-72, and/or from about 0.01 to about 3.00 wt % of at least one silicone known by the INCI designator Dimethicone. The composition has a pH value in the range from about 4.0 to about 5.0.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object underlying the present disclosure was to provide compositions for treating keratin fibers, particularly hair, with which the adhesion of dyes to the fibers is strengthened so that the fastness of the color can be preserved for a long period. Ideally, the cosmetic compounds provided should be mild and well tolerated by the skin, lend the hair improved visual and haptic properties besides preserving the color, and should not stress the hair or scalp.

Surprisingly, it was found that the aforementioned objects are solved to an exceptional degree by the use of compositions for treating keratinous fibers that contain at least one alum and at least one salt which comprises a bivalent cation. The use of these compositions on colored keratin fibers—particularly dyed and/or bleached hair—led to a long-lasting, marked stabilization of the changed color. The color protection effect was particularly sustained on keratin fibers whose color had been changed by oxidative compositions. Color fidelity on the “b-axis” in the LAB color space (EN ISO 11664-4) was particularly enhanced by the use of the compositions as contemplated herein (shift towards yellow and blue reduced).

A first object as contemplated herein is therefore a composition for treating keratinous fibers containing—relative to its weight—

a) from about 0.01 to 4.00 wt % of at least one alum and
b) from about 0.01 to 5.00 wt % of at least one salt which comprises a bivalent cation.

In the context as contemplated herein, the terms keratinous and keratin-containing fibers are understood to refer to furs, wool, feathers, and particularly human hair. Although the compositions as contemplated herein are primarily designed for preserving the color of and/or caring for keratin fibers, theoretically there is nothing to prevent them from being used in other areas as well.

Suitable compositions for treating keratinous fibers—particularly hair—include for example hair cleaning substances such as shampoos, hair care products such as conditioners, rinses or hair care sprays, and styling substances such as hair gels, hairsprays or hair waxes. Hair cleaning substances and/or hair care products are particularly preferred.

Preferred compositions as contemplated herein preferably contain the active ingredients a) and b) in a cosmetically acceptable carrier. As contemplated herein, this is preferably understood to mean an aqueous or aqueous-alcoholic carrier.

The cosmetic carrier preferably contains at least about 50 wt %, more preferably at least about 60 wt %, particularly preferably at least about 70 wt % and most particularly preferably at least about 75 wt % water. The cosmetic carrier may further contain about 0.01 to 40 wt %, preferably from about 0.05 to about 30 wt % and particularly from about 0.1 to about 20 wt % of at least one alcohol.

Suitable alcohols are for example ethanol, ethyl diglycol, 1-propanol, 2-propanol, isopropanol, 1,2-propylene glycol, 1,3-propylene glycol, glycerin, diglycerin, triglycerin, 1-butanol, 2-butanol, 1,2-butanediol, 1,3-butanediol, 1,4-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, phenoxy ethanol or mixtures of these alcohols.

The water-soluble alcohols are particularly preferred. More particularly preferred are ethanol, 1,2-propylene glycol, glycerin, benzyl alcohol and/or phenoxy ethanol and mixtures of these alcohols.

It was found that the color protection effect of the compositions as contemplated herein can be intensified by additional factors. These factors preferably include the weight ratio of components a) and b) in the compositions as contemplated herein, the pH value of said compositions and the careful selection and combination of particularly preferred alums and salts b).

In a first preferred embodiment, inventive compositions are preferred in which the weight ratio of the at least one alum a) to the at least one salt b) is in a range from about 4:1 to about 1:3, preferably from about 3.5:1 to about 1:2.5, particularly preferably from about 3:1 to about 1:2 and particularly from about 2.5:1 to about 1:1.5.

In a second preferred embodiment, inventive compositions are preferred which have a pH value in the range from about 4.0 to about 5.0, more preferably from about 4.1 to about 4.9, particularly preferably from about 4.2 to about 4.8 and particularly from about 4.3 to about 4.7. Compositions as contemplated herein that are formulated in this pH range are particularly mild, well tolerated by the skin and scalp, and impart a particular gloss and greater volume to keratin fibers, particularly hair.

The term “alums” is understood to refer preferably to metal sulphate salts—and/or -double salts having general formula MIMIII(SO4)2×12H2O, in which
MI preferably stands for an alkaline metal ion, an ammonium ion or a guanidinium ion,
MIII preferably stands for an aluminum, gallium, indium, titanium, vanadium, chromium, manganese, iron, cobalt or rhodium ion,
   and which preferably crystallize with 12 molecules of water of crystallization.

Particularly preferred alums for the compositions as contemplated herein correspond to the formula MIAl(SO4)2×12H2O, in which MI may preferably stand for an alkaline metal ion, particularly a sodium ion or a potassium ion, an ammonium ion or a guanidinium ion. Particularly preferred for the color protection effect of the compositions as contemplated herein are NaAl(SO4)2×12H2O, KAl(SO4)2×12H2O, NH4Al(SO4)2×12H2O and mixtures of these alums.

In a particularly preferred embodiment, the compositions as contemplated herein therefore contain an alum a) of formula MIAl(SO4)2×12H2O in which MI stands for a potassium, sodium, ammonium or guanidinium ion.

In a further particularly preferred embodiment, the compositions as contemplated herein contain—relative to their weight—from about 0.05 to about 3.50 wt %, preferably from about 0.10 to about 3.00 wt %, more preferably from about 0.15 to about 2.50 wt %, particularly preferably from about 0.20 to about 2.25 wt % and most particularly preferably about 0.25 to about 2.00 wt % of at least one alum a), preferably a potassium, sodium or ammonium alum having the abovementioned formula.

Suitable salts b) may be selected from organic and/or inorganic salts. Particularly suitable bivalent cations may preferably be chosen within these salts from alkaline earth metal cations and from copper, zinc and/or iron(II) cations. Most particularly preferred are alkaline earth metal cations and particularly preferably calcium and magnesium cations.

Particularly suitable organic anions may preferably be chosen within these salts from acetate, lactate, succinate, citrate, tartrate, malate, maleate, oxalate and/or glycolate ions. Most particularly preferred are acetate, lactate and/or citrate salts with the aforementioned cations.

Most particularly preferred organic salts b) are calcium lactate, calcium citrate, calcium acetate, magnesium lactate, magnesium citrate and/or magnesium acetate. Particularly preferred are calcium lactate and/or magnesium lactate. Particularly suitable inorganic anions may be chosen within these salts from halide, sulphate, phosphate and/or carbonate ions. Most particularly preferred are sulphate and/or halide ions such as chloride and bromide ions.

Particularly preferred inorganic salts b) are calcium chloride, calcium sulphate, magnesium chloride and/or magnesium sulphate. The percentage by weight of the at least one salt b) in the total weight of the compositions as contemplated herein is preferably from about 0.02 to about 4.50 wt %, more preferably from about 0.05 to about 4.00 wt %, particularly preferably about 0.10 to about 3.50 wt % and most particularly about 0.20 to about 3.00 wt %.

In a third particularly preferred embodiment, compositions as contemplated herein are characterized in that they contain an organic or inorganic salt of copper, zinc, iron(II), calcium and/or magnesium as salt b).

It was found that particularly good color protection results can be obtained in the compositions as contemplated herein with organic salts of copper, zinc, iron(II), calcium and/or magnesium, and the most marked effects were achieved with the alkaline earth metal salts of lactic acid, particularly with calcium lactate and/or magnesium lactate. Consequently, in a further particularly preferred embodiment the compositions as contemplated herein contain calcium lactate and/or magnesium lactate, particularly calcium lactate as salt b).

It is also possible as contemplated herein that the aforementioned salts b) are only formed in the compositions as contemplated herein until an acid and a salt are added. For example, calcium lactate may be added to the compositions as contemplated herein as a salt. However, it is also possible to add lactic acid and for example calcium hydroxide or a calcium halide to the compositions. For some embodiments, it has proven particularly preferable to add an excess of lactic acid first, and then to adjust the pH value to the preferred range described previously using calcium hydroxide. This method sequence has been shown to improve the stability of the compositions.

Particularly suitable compositions as contemplated herein for treating keratinous fibers are hair shampoos, hair rinses and/or hair conditioners which may contain further ingredients usual for the respective compositions besides the ingredients described above. Such preferred usual ingredients in the aforementioned compositions include surfactants.

Further usual ingredients and examples of standard formulations of the aforementioned hair treatment compositions are presented for example in the monograph by Karlheinz Schrader: “Grundlagen and Rezepturen der Kosmetika” [Cosmetic principles and recipes], 2nd edition (1989), Hüthig Buch Verlag GmbH Heidelberg, pages 676-848.

In a further preferred embodiment, compositions as contemplated herein are characterized in that that contain from about 0.05 to about 20 wt %—relative to their weight—of at least one surfactant.

As a rule, hair dyes can change, typically during or after hair cleaning activities. Contact between the hair and water together with surfactants, but also the acts of massaging a shampoo into the hair, towelling the hair dry after rinsing the shampoo out, or the heat of a hairdryer in the subsequent drying process can all impair the adhesion of the hair color and cause undesirable color change and/or loss of the glossy appearance of the artificially created hair color. It is therefore particularly desirable that the process for cleaning dyed hair may be carried out particularly gently.

It was found that the process for cleaning dyed hair can be carried out very gently if the composition as contemplated herein is prepared as a hair shampoo or as a hair conditioner which is applied immediately after the hair is cleaned.

In a further preferred embodiment, the composition as contemplated herein is therefore packaged as a hair shampoo. In the context of this embodiment, it is particularly preferred if the composition as contemplated herein contains from about 1.00 to about 20.00 wt % of at least one anionic surfactant—relative to its weight.

The group of particularly preferred anionic surfactants includes for example:
ether carboxylic acids with formula R—O—(CH2-CH2O)x-CH2-COOH, in which R is a linear or branched chain, saturated or unsaturated alkyl group with 8 to 30 C atoms, and x=0 or 1 to 16,
acyl glutamates and/or (acyl)isethionates with 8 to 24 C atoms in the acyl group,
mono- and/or -dialkyl esters of sulphosuccinic acid with 8 to 24 C atoms in the alkyl group and monoalkylpolyoxyethyl esters of sulphosuccinic acid with 8 to 24 C atoms in the alkyl group and 1 to 6 oxyethyl groups, and/or
alkyl sulphates and/or alkyl polyglycol ether sulphate salts having formula R—(OCH2-CH2)x-OSO3-X+, in which R is preferably a linear or branched chain, saturated or unsaturated alkyl group with 8 to 30 C atoms, x is the integer 0 or 1 to 12, and X is an alkaline, alkaline earth, ammonium or alkanol amine ion.

Particularly preferred anionic surfactants are straight chain or branched chain alkyl ether sulphates having the aforementioned formula, which contain an alkyl radical with 8 to 18, particularly 10 to 16 C atoms and 1 to 6 and particularly 2 to 4 ethylene oxide units.

Particularly preferred are the sodium, magnesium and/or triethanolamine salts of linear or branched lauryl, tridecyl and/or myristyl sulphates with a degree of ethoxylation from 2 to 4.

The percentage by weight of the anionic surfactants in the total weight of the compositions as contemplated herein which are prepared as hair shampoo is from about 1.00 to about 20.00 wt %. More preferred is a percentage by weight from about 2.00 to about 18.00 wt %, particularly preferably from about 3.00 to about 17.50 wt % and especially from about 4.00 to about 15.00 wt %.

In a further preferred embodiment, the composition as contemplated herein is prepared as a hair rinse or hair conditioner. In the context of this embodiment, it is particularly preferable if the composition as contemplated herein contains about 0.10 to about 10.00 wt % of at least one cationic surfactant—relative to its weight. Suitable cationic surfactants are for example quaternary ammonium compounds, esterquats and/or amidoamines.

Preferred quaternary ammonium compounds are ammonium halides, particularly chlorides and bromides, such as alkyltrimethyl ammonium chlorides, dialkyldimethyl ammonium chlorides and trialkylmethyl ammonium chlorides, e.g., cetyltrimethyl ammonium chloride, stearyltrimethyl ammonium chloride, distearyldimethyl ammonium chloride, lauryldimethyl ammonium chloride, lauryldimethyl benzylammonium chloride and tricetylmethyl ammonium chloride, and the imidazolium compounds known by the INCI designators Quaternium-27, Quaternium-83 and Quaternium-87. The alkyl chains in the surfactants described above preferably have 10 to 18 carbon atoms.

Esterquats are substances which have both at least one ester function and at least one quaternary ammonium group as a structural element. Preferred esterquats are quaternised ester salts of fatty acids with triethanolamine, quaternized ester salts of fatty acids with diethanol alkylamines and quaternized ester salts of fatty acids with 1,2-dihydroxypropyl dialkylamines. Such products are marketed for example under the trade names Stepantex®, Dehyquart®, Armocare® and Quartamin®.

The alkylamidoamines are usually prepared by amidation of natural or synthetic fatty acids and fatty acid cuts with dialkylaminoamines. Dimethylaminopropyl stearamide, which is available commercially with the name Tegoamid® S 18 represents a particularly suitable compound from this substance group.

The percentage by weight of the at least one cationic surfactant in the total weight of the compositions as contemplated herein that are prepared as hair rinse or hair conditioner is from about 0.10 to about 10.00 wt %. More preferable is a percentage by weight from about 0.15 to about 8.00 wt %, particularly preferably from about 0.20 to about 7.50 wt % and especially from about 0.25 to about 5.00 wt %. It has been found that the care effect of the compositions as contemplated herein may be enhanced still further if at least one special active hair conditioning substance, preferably at least one silicone and/or at least one cationic conditioning polymer, is added to them.

In a further preferred embodiment, compositions as contemplated herein are characterized in that they contain from about 0.01 to about 3.00 wt % of at least one silicone relative to their weight. Still another preferred embodiment of the hair treatment compositions as contemplated herein is characterized in that they contain from about 0.01 to about 3 wt % of at least one cationic polymer relative to their weight.

Suitable silicones may be selected from:

a) polyalkylsiloxanes, polyarylsiloxanes, polyalkylarylsiloxanes which are volatile or non-volatile, straight chain, branched or cyclic, crosslinked or non-crosslinked;
b) polysiloxanes with a general structure which contains one or more organofunctional groups selected from:
a) substituted or unsubstituted aminated groups;
b) (per)fluorinated groups;
c) thiol groups;
d) carboxylate groups;
e) hydroxylated groups;
f) alkoxylated groups;
g) acyloxyalkyl groups;
h) amphoteric group;
i) bisulphite groups;
j) hydroxyacylamino groups;
k) carboxy groups;
l) sulphonic acid groups; and
m) sulphate or thiosulphate groups;
   c) linear polysiloxane(A)-polyoxyalkylene(B) block copolymers of type (A-B)n where n>3;
   d) grafted silicone polymers with organic base structure that contains no silicone and which consist of an organic primary chain formed by organic monomers that contain no silicone, to which at least one polysiloxane macromer has been grafted in the chain and optionally to at least one end of the chain;
   e) grafted silicone polymers with polysiloxane base structure to which gave been grafted organic monomers that contain no silicone and which have a polysiloxane primary chain to which at least one organic macromer has been grafted in the chain and optionally to at least one of the free ends thereof;
   f) or mixtures thereof.

Suitable cationic polymers are for example:
quaternized cellulose derivatives, such as are available commercially under the names Celquat® and Polymer JR®,
hydrophobically modified cellulose derivatives, for example the cationic polymers marketed with the trade name SoftCat®,
cationic alkylpolyglycosides,
cationized honey, for example the commercial product Honeyquat® 50,
cationic guar derivatives, particularly such as the products marketed under the trade names Cosmedia®Guar N-Hance® and Jaguar®,
polymeric dimethyldiallyl ammonium salts and copolymers thereof with esters and amides of acrylic acid and methacrylic acid. The products that are available commercially with the designators Merquat®100 (poly(dimethyldiallyl ammonium chloride)) and Merquat®550 (dimethyldiallyl ammonium chloride-acrylamide copolymer) are examples of such cationic polymers,
copolymers of vinylpyrrolidone with quaternized derivatives of dialkylamino alkylacrylate and -methacrylate, such as for example vinylpyrrolidone-dimethyl aminoethyl methacrylate copolymer quaternized with diethylsulphate. Such compounds are available commercially under designators Gafquat®734 and Gafquat®755,
vinylpyrrolidone-vinylimidazolium methochloride copolymers, such as are offered commercially under the designations Luviquat® FC 370, FC 550, FC 905 and HM 552,
quaternized polyvinyl alcohol, and the polymers known by the designators
Polyquaternium 2, Polyquaternium 17, Polyquaternium 18, Polyquaternium-24, Polyquaternium 27, Polyquaternium-32, Polyquaternium-37, Polyquaternium 74 and Polyquaternium 89.

Preferred cationic polymers are cationic polysaccharide polymers such as quaternized cellulose polymers, hydrophobically modified cationic cellulose derivatives and/or cationic guar derivatives which are particularly preferably chosen from the Guar Hydroxypropyltrimonium Chloride polymers known by the INCI designators Polyquaternium-10, Polyquaternium-37, Polyquaternium-67 and/or Polyquaternium-72. It was found that the vibrant color of dyed keratin fibers can be preserved particularly well by treatment with the compositions as contemplated herein if the compositions contain at least one cationic polymer, preferably a cationic polysaccharide and at least one silicone as active hair conditioning agents. With the addition of these special care substances, it was also possible to improve the haptic properties such as texture and smoothness of the dyed hair.

In a further preferred embodiment, the compositions as contemplated herein therefore additionally contain a cationic polymer—particularly a cationic polysaccharide—and a silicone as active hair conditioning agents.

In principle, the compositions as contemplated herein can be used on keratinous fibers that that have be dyed using permanent, semipermanent or temporary coloring systems. However, temporary coloring systems are designed to be washed out and/or fade over a period of time, so the compositions as contemplated herein are particularly suitable for application to keratinous fibers that have been dyed using permanent or oxidative hair coloring compositions.

In a further preferred embodiment, therefore, compositions as contemplated herein that are used before or after an oxidative treatment (coloring) of keratinous fibers are particularly preferred.

Besides the aforementioned ingredients, the compositions as contemplated herein may also contain further substances which lend them additional advantageous properties or enhance such properties. Examples of such further substances are natural and/or mineral oil, grease or wax components (excluding silicones), vitamins and/or protein hydrolysates.

The group of suitable protein hydrolysates includes product mixtures that can be obtained by the acid, alkaline or enzymatically catalyzed breakdown of proteins. Protein hydrolysates of vegetable, animal and/or marine origin can be used. Animal protein hydrolysates are for example protein hydrolysates from elastin, collagen, keratin, silk and milk protein, and may also be present in the form of salts. Such products are marketed under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda). Protein hydrolysates of vegetable origin, e.g., soya, almond, rice, pea, potato and wheat protein hydrolysates. Such products are available commercially under the trade names Gluadin® (Cognis), DiaMin® (Diamalt), Lexein® (Inolex) and Crotein® (Croda) for example.

Cationized protein hydrolysates may also be used, in which case the base protein hydrolysate may be of animal origin, for example from collagen, milk or keratin, vegetable origin, for example from wheat, maize, rice, potatoes, soya or almonds, from marine life forms, from fish collagen or seaweed for example, or from bioengineered protein hydrolysates. The protein hydrolysates on which the cationic derivatives are based may be obtained from the corresponding proteins by chemical, particularly alkaline or acid hydrolysis, by enzymatic hydrolysis and/or a combination of both hydrolysis methods. The hydrolyse of proteins typically yields a protein hydrolysate with a molecular weight distribution from about 100 Dalton up to several thousand Dalton. Preferred are cationic protein hydrolysates of which the protein component from which they are derived a molecular weight from 100 up to 25000 Dalton, preferably 250 to 5000 Dalton. In addition, the term cationic protein hydrolysates is understood to include quaternized amino acids and their mixtures. The quaternization of the protein hydrolysates or the amino acids is often carried out using quaternary ammonium salts such as N,N-dimethyl-N-(n-alkyl)-N-(2-hydroxy-3-chloro-n-propyl)-ammonium halides. The cationic protein hydrolysates may also be further derivatized. Typical examples of the cationic protein hydrolysates and derivatives include the following products which are known by their INCI designators and are available commercially: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The one or more protein hydrolysates may be used in the compositions as contemplated herein preferably in the quantities of from about 0.01 to about 5 wt %, more preferably from about 0.025 to about 3 wt % and particularly from about 0.05 to about 2 wt %, relative to the total weight thereof.

The list of suitable vitamins preferably includes the following vitamins, provitamins and vitamin precursors and derivatives thereof:
Vitamin A: The group of substances which are classified together as vitamin A includes retinol (vitamin A1) and 3,4-didehydroretinol (vitamin A2). B-carotine is the provitamin of retinol. Vitamin A acid and esters thereof, vitamin A aldehyde and vitamin A alcohol and its esters such as the palmitate and the acetate are suitable for use as the Vitamin A component.
Vitamin B: The vitamin B group or vitamin B complex includes among others
   Vitamin B1 (thiamine)
   Vitamin B2 (riboflavin)
   Vitamin B3. This designation is often used to identify the compounds nicotinic acid and nicotinic acid amide (niacin amide).
   Vitamin B5 (pantothenic acid and panthenol). From this group, panthenol is used for preference. Usable derivatives of panthenol are particularly the ester and ethers of panthenol, pantolactone and cationically derivatized panthenols. Individual representatives are for example panthenol triacetate, panthenol monoethyl ether and the monoacetate thereof and cationic panthenol derivatives.
   Vitamin B6 (pyridoxine and pyridoxamine and pyridoxal).
Vitamin C (ascorbic acid): Use in the form of the palmitic acid ester, the glucosides or phosphates may be preferred. Use in combination with tocopherols may also be preferred.
Vitamin E (tocopherols, particularly α-tocopherol).
Vitamin F: The term “Vitamin F” is typically understood to include essential fatty acids, particularly linoleic acid, linolenic acid and arachidonic acid.
Vitamin H: Vitamin H refers to the compound (3aS,4S, 6aR)-2-oxohexahydrothienol[3,4-d]-imidazole-4-valerian acid, for which the trivial name biotin has now gained acceptance.

The compositions as contemplated herein may preferably contain vitamins, provitamins and vitamin precursors from groups A, B, E and H. Particularly preferred are nicotinic acid amide, biotin, pantolactone and/or panthenol. Vitamins, vitamin derivatives and/or vitamin precursors may be used in the compositions as contemplated herein preferably in a quantity from about 0.001 to about 2 wt %, particularly preferably from about 0.005 to about 1 wt % and most particularly from about 0.01 to about 0.5 wt %, relative to the total weight thereof.

Suitable natural (vegetable) oils are usually understood to include triglycerides and mixtures of triglycerides. Preferred natural oils are coconut oil, (sweet) almond oil, walnut oil, peach kernel oil, apricot kernel oil, avocado oil, tea tree oil, soya oil, sesame oil, sunflower oil, tsubaki oil, evening primrose oil, rice bran oil, palm kernel oil, mango kernel oil, cuckoo flower oil, safflower oil, macadamia nut oil, grape seed oil, amaranth seed oil, argan oil, bamboo oil, olive oil, wheatgerm oil, pumpkin seed oil, mallow oil, hazelnut oil, safflower oil, canola oil, camellia sasanqua oil, jojoba oil, rambutan oil, cocoa butter and/or shea butter. Mineral oils that are usable include in particularly mineral oils, paraffin oils and isoparaffin oils as well as synthetic hydrocarbons. An example of a usable hydrocarbon is for example the commercially available product 1,3-di-(2-ethylhexyl)-cyclohexane (Cetiol® S). A dialkylether may also serve as an oil component.

Usable dialkylethers particularly include di-n-alkylethers with a total of 12 to 36 C atoms, particularly 12 to 24 C atoms, such as di-n-octylether, di-n-decylether, di-n-nonylether, di-n-undecylether, di-n-dodecylether, n-hexyl-n-octylether, n-octyl-n-decylether, n-decyl-n-undecylether, n-undecyl-n-dodecylether and n-hexyl-n-undecylether as well as di-tert.-butylether, di-iso-pentylether, di-3-ethyldecylether, tert.-butyl-n-octylether, iso-pentyl-n-octylether and 2-methylpentyl-n-octylether. Particularly preferable is di-n-octylether, which is available commercially with the name Cetiol® OE. The term fats is understood to include fatty acids, fatty alcohols as well as natural synthetic waxes, which may be present in solid form or also in liquid form in aqueous dispersion. Linear and/or branched chain, saturated and/or unsaturated fatty acids with 6-30 carbon atoms are usable as fatty acids. Preferred are fatty acids with 10-22 carbon atoms. These would include for example the isostearic acids such as the products available commercially as Emersol® 871 and Emersol® 875, and isopalmitic acids available commercially as the product Edenor® IP 95, and all other fatty acids that are marketed with the trade names Edenor® (Cognis). Other typical examples of such fatty acids are caproic acid, caprylic acid, 2-ethylhexane acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachic acid, gadoleic acid, behenic acid and erucic acid and technical mixtures thereof.

Particularly preferable are usually the fatty acid cuts that can be obtained from coconut oil or palm oil; more particularly, the use of stearic acid is usually preferred. Substances that may be used as fatty alcohols are saturated, mono- or polyunsaturated, branched chain or unbranched fa fatty alcohols with C6-C30, preferably C10-C22 and most particularly preferably C12-C22 carbon atoms. It is possible to use for example decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, caprin alcohol, linoleyl alcohol, linolenyl alcohol and behenyl alcohol, as well as the guerbet alcohols thereof, wherein is should be noted that this list is exemplary and non-limiting in nature. However, the fatty alcohols are preferably obtained from natural fatty acids, wherein it may typically be assured that they are recovered from the esters of the fatty acids by reduction. Also usable as contemplated herein are fatty alcohol cuts which are produced by reduction of naturally occurring triglycerides, such as beef tallow, palm oil, peanut oil, rapeseed oil, cottonseed oil, soya oil, sunflower oil and linseed oil or from the fatty acid esters created from the products of transesterification thereof with corresponding fatty alcohols, which thus represent a mixture of various fatty alcohols. Such substances are available for purchase on the market for example under the names Stenol®, e.g., Stenol® 1618 or Lanette®, e.g., Lanette® 0 or Lorol®, e.g., Lorol® C8, Lorol® C14, Lorol® C18, Lorol® C8-18, HD-Ocenol®, Crodacol®, e.g., Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Of course, lanolin alcohols such as may be purchased for example under the names Corona®, White Swan®, Coronet® or Fluilan® are also usable as contemplated herein. Solid paraffins or isoparaffins, carnauba waxes, beeswaxes, candelilla waxes, ozokerite, ceresin, spermaceti, sunflower wax, fruit waxes such as apple wax or citrus wax, microwaxes from PE- or PP are usable as natural or synthetic waxes. Such waxes may be obtained for example from Kahl & Co., Trittau.

Other fatty substances include for example
Ester oils. Ester oils are understood to include the esters of C6-C30-fatty acids with C2-C30-fatty alcohols. Preferred are the monoester of fatty acids with alcohols having 2 to 24 C atoms. Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleinic acid, behenic acid and erucic acid and technical mixtures thereof. Examples of the fatty alcohol components in the esteroils are isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol and technical mixtures thereof. Particularly preferred are isopropyl myristate (Rilanit® IPM), isononanoic acid-C16-18-alkyl ester (Cetiol® SN), 2-ethylhexyl palmitate (Cegesoft® 24), Stearinacid-2-ethylhexylester (Cetiol® 868), cetyl oleate, glycerin tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexylester (Cetiol® A), di-n-butyl adipate (Cetiol® B), myristyl myristate (Cetiol® MM), cetearyl isononanoate (Cetiol® SN), oleic acid decylester (Cetiol® V).
Dicarboxylic acid esters such as di-n-butyl adipate, di-(2-ethylhexyl)-adipate, di-(2-ethylhexyl)-succinate and di-isotridecylacetate and diol esters such as ethylene glycol-dioleate, ethylene glycol-di-isotridecanoate, propylene glycol-di(2-ethylhexanoate), propylene glycol-di-isostearate, propyleneglycol-di-pelargonate, butanediol-di-isostearate, neopentyl glycol dicaprylate,
symmetrical, asymmetrical or cyclic esters of carbonic acid with fatty alcohols,
glycerin carbonate or dicaprylyl carbonate (Cetiol® CC),
ethoxylated or non-ethoxylated mono-, di-, and trifatty acid esters of saturated and/or unsaturated linear and/or branched chain fatty acids with glycerin, such as Monomuls® 90-018, Monomuls® 90-L12, Cetiol® HE or Cutina® MD.

The weight of the oil, wax and/or fat components as a percentage of the total weight of the compositions as contemplated herein is preferably from about 0.01 to about 10 wt %, particularly preferably from about 0.025 to about 7.5 wt % and particularly from about 0.05 to about 5 wt %.

In a fourth preferred embodiment, compositions as contemplated herein are characterized in that they contain—relative to their total weight—:

a) from about 0.05 to about 3.50 wt %, preferably from about 0.10 to about 3.00 wt %, more preferably from about 0.15 to about 2-50 wt %, particularly preferably from about 0.20 to about 2.25 wt % and particularly preferably from about 0.25 to about 2.00 wt % of at least one alum with formula MIMIII(SO4)2×12H2O, in which
MI preferably stands for an alkaline metal ion, an ammonium ion or a guanidinium ion, and
MIII preferably stands for an aluminum, gallium, indium, titanium, vanadium, chromium, manganese, iron, cobalt or rhodium ion,
   b) from about 0.02 to about 4.50 wt %, more preferably from about 0.05 to about 4.00 wt %, particularly preferably about 0.10 to 3.50 wt % and particularly from about 0.20 to about 3.00 wt % of at least one organic copper, zinc, iron(II), calcium and/or magnesium salt, particularly a lactic acid salt,
   c) from about 2.00 to about 18.00 wt %, particularly preferably from about 3.00 to about 17.50 wt % and particularly from about 4.00 to about 15.00 wt % of at least one anionic surfactant and/or from about 0.15 to about 8.00 wt %, particularly preferably from about 0.20 to about 7.50 wt % and particularly from about 0.25 to about 5.00 wt % of at least one cationic surfactant, and
   d) from about 0.01 to about 3.00 wt %, more preferably from about 0.05 to about 2.50 wt % and particularly from about 0.10 to about 2.00 wt % of at least one cationic polymer and/or from about 0.01 to about 3.00 wt %, more preferably from about 0.05 to about 2.50 wt % and particularly from about 0.10 to about 2.00 wt % of at least one silicone, and have a pH value in the range from about 4.0 to about 5.0, more preferably from about 4.1 to about 4.9, particularly preferably from about 4.2 to about 4.8 and particularly from about 4.3 to about 4.7.

In the context of this embodiment, particularly preferred compositions as contemplated herein are those which contain

a) from about 0.05 to about 3.50 wt %, preferably from about 0.10 to about 3.00 wt %, more preferably from about 0.15 to about 2.50 wt %, particularly preferably from about 0.20 to about 2.25 wt % and most particularly preferably from about 0.25 to about 2.00 wt % of at least one alum of formula MIAl(SO4)2×12H2O, in which MI preferably stands for an alkaline metal ion, particularly a sodium ion or a potassium ion, an ammonium ion or a guanidinium ion,
b) from about 0.02 to about 4.50 wt %, more preferably from about 0.05 to about 4.00 wt %, particularly preferably from about 0.10 to about 3.50 wt % and most particularly from about 0.20 to about 3.00 wt % calcium lactate and/or magnesium lactate,
c) from about 2.00 to about 18.00 wt %, particularly preferably from about 3.00 to about 17.50 wt % and particularly from about 4.00 to about 15.00 wt % of at least one alkyl sulphate and/or alkylpolyglycol ether sulphate salt having formula R—(OCH2-CH2)x-OSO3-X+, in which R preferably represents a linear or branched chain, saturated or unsaturated alkyl group with 8 to 30 C atoms, x is 0 or 1 to 12, and X represents an alkaline, alkaline earth, ammonium or alkanol amine ion, and
d) from about 0.01 to about 3.00 wt %, more preferably from about 0.05 to about 2.50 wt % and particularly from about 0.10 to about 2.00 wt % of at least one of the cationic polymers known by the INCI designators Polyquaternium-10, Guar Hydroxypropyltrimonium Chloride, Polyquaternium-37, Polyquaternium-67 and/or Polyquaternium-72 and/or from about 0.01 to about 3.00 wt %, more preferably from about 0.05 to about 2.50 wt % and most particularly from about 0.10 to about 2.00 wt % of at least one silicone known by the INCI designator Dimethicone.

In the context of this embodiment, particularly preferred compositions as contemplated herein are those which contain

a) from about 0.05 to about 3.50 wt %, preferably from about 0.10 to about 3.00 wt %, more preferably from about 0.15 to about 2.50 wt %, particularly preferably from about 0.20 to about 2.25 wt % and most particularly preferably from about 0.25 to about 2.00 wt % of at least one alum of formula MIAl(SO4)2×12H2O, in which MI preferably stands for an alkaline metal ion, particularly a sodium ion or a potassium ion, an ammonium ion or a guanidinium ion,
b) from about 0.02 to about 4.50 wt %, more preferably from about 0.05 to about 4.00 wt %, particularly preferably from about 0.10 to about 3.50 wt % and most particularly from about 0.20 to about 3.00 wt % calcium lactate and/or magnesium lactate,
c) from about 0.15 to about 8.00 wt %, particularly preferably from about 0.20 to about 7.50 wt % and particularly from about 0.25 to about 5.00 wt % cetyl trimethyl ammonium chloride, stearyl trimethyl ammonium chloride, behen trimethyl ammonium chloride, distearyl dimethyl ammonium chloride, lauryl dimethyl ammonium chloride, lauryl dimethylbenzyl ammonium chloride and/or tricetylmethyl ammonium chloride, and
d) from about 0.01 to about 3.00 wt %, more preferably from about 0.05 to about 2.50 wt % and particularly from about 0.10 to about 2.00 wt % of at least one of the cationic polymers known by the INCI designators Polyquaternium-10, Guar Hydroxypropyltrimonium Chloride, Polyquaternium-37, Polyquaternium-67 and/or Polyquaternium-72 and/or from about 0.01 to about 3.00 wt %, more preferably from about 0.05 to about 2.50 wt % and most particularly from about 0.10 to about 2.00 wt % of at least one silicone known by the INCI designator Dimethicone.

The compositions as contemplated herein may contain one or more compounds from the following groups as further optional ingredients:
nonionic and/or amphoteric surfactants,
anti-dandruff agents,
pearl shine and/or opacifiers.

The group of suitable nonionic surfactants includes for example:
C8-C30-fatty acid mono- and diesters from adducts from 1 to 30 Mol ethylene oxide to glycerin,
amine oxides,
adducts from about 2 to about 50 Mol ethylene oxide and/or 0 to about 5 Mol propylene oxide to linear and branched chain fatty alcohols with 8 to 30 C atoms, to fatty acids with 8 to 30 C atoms and to alkyl phenols with 8 to 15 C atoms in the alkyl group,
sorbitan fatty acid esters and adducts from ethylene oxide to sorbitan fatty acid esters such as polysorbates,
fatty acid alkanol amides,
sugar fatty acid esters and adducts from ethylene oxide to sugar fatty acid esters,
adducts from ethylene oxide to fatty acid alkanol amides and fatty amines and/or
alkyl polyglucosides having general formula RO-[G]x, in which R stands for an alkyl- and/or alkenyl radical with 4 to 22 C atoms, G stands for a sugar radical with 5 or 6 C atoms, and x stands for integers from 1 to 10. Particularly suitable alkyl polyglucosides are obtained from aldoses and/or ketoses with 5 or 6 carbon atoms, preferably from glucose. Radical R stands particularly preferably for an alkyl radical with 6 to 20 and most particularly 8 to 18 carbon atoms. In the general formula RO-[G][x] index number x stands for degree of oligomerization (DP), that is to say the distribution of mono- and oligoglycosides. Index number x preferably has a value in the range from 1 to 6, particularly preferably in the range from 1 to 3, wherein the number is not necessarily an integer, but may also be a fraction which can be determined analytically. Particularly preferred alkyl polyglucosides have a degree of oligomerization between about 1.2 and about 1.5. Particularly suitable alkyl polyglucosides are known and are available commercially from various suppliers with the INCI names Decyl Glucoside, Lauryl Glucoside and Coco Glucoside.

Particularly suitable nonionic surfactants/emulsifiers are alkyl polyglucosides, more particularly alkyl polyglucosides based on hardened C10-14 coconut alcohol with a DP from 1-3, such as are available commercially with the INCI designation “Coco-Glucoside”. The one or more nonionic surfactant(s) is (are) used in the compositions as contemplated herein preferably in a quantity from about 0.10 to about 15.00 wt %, more preferably from about 0.25 to about 12.50 wt %, particularly preferably from about 0.40 to about 10.00 wt % and most particularly from about 0.50 to about 5.00 wt % relative to the total weight thereof.

The group of suitable amphoteric and/or zwitterionic surfactants includes the “betaines” such as the N-Alkyl-N,N-dimethylammonium glycinates, for example coconut alkyl dimethylammonium glycinate, N-Acyl-aminopropyl-N,N-dimethylammoniumglycinate, for example coconut acylaminopropyl-dimethylammonium glycinate, and 2-Alkyl-3-carboxymethyl-3-hydroxyethyl-imidazoline, each having 8 to 18 C atoms in the alkyl or acyl group, and coconut acyl aminoethyl-hydroxyethyl-carboxymethyl glycinate. Regarding the amphoteric surfactants, surface active compounds are understood to be those which are able to form inner salts. Examples of suitable amphoteric surfactants are N-Alkylglycines, N-Alkylpropionic acids, N-Alkylamino butyric acids, N-Alkylimino dipropionic acids, N-Hydroxyethyl-N-alkylamidopropylglycines, N-Alkyltaurines, N-Alkylsarcosines, 2-Alkylamino propionic acids and Alkylamino acetic acids, each having about 8 to 24 C atoms in the alkyl group.

Typical examples of amphoteric and zwitterionic surfactants are alkyl betaines, alkyl amidobetaines, aminopropionates, aminoglycinates, imidazolinium betaines and sulphobetaines. Particularly preferred amphoteric surfactants are N-coconut alkyl aminopropionate, coconut acyl aminoethyl aminopropionate, the fatty acid amide derivate known with INCI designator Cocamidopropyl Betaine, and C12-C18-acyl sarcosine. The total quantity of amphoteric and/or zwitterionic surfactants in the compositions as contemplated herein is preferably from about 1.00 to about 15.00 wt %, more preferably from about 1.25 to about 12.50 wt %, particularly preferably from about 1.50 to about 10.00 wt % and most particularly from about 1.75 to about 7.50 wt %, wherein the percentages relate to the total weight of the compositions.

Anti-dandruff substances may be used in the compositions as contemplated herein preferably in quantities from about 0.01 to about 10 wt %, more preferably from about 0.025 to about 7.5 wt %, particularly preferably from about 0.05 to about 5 wt % and most particularly from about 0.075 to about 3 wt % relative to the total weight thereof. Suitable anti-dandruff substances may be chosen from Piroctone Olamine, Climbazol, Zinc Pyrithione, Ketoconazole, Salicylic acid, Sulphur, Selenium sulphide, Tar preparations, Undecenoic acid derivatives, Burdock Root Extracts, Cottonwood Extracts, Stinging Nettle Extracts, Walnut Shell Extracts, Birch Extracts, Willow Bark Extracts, Rosemary Extracts and/or Arnica Extracts. Preferred are Climbazol, Zinc Pyrithione, Piroctone Olamine and/or Salicylic Acid. Particularly preferred is zinc pyrithione.

Suitable opacifiers and/or pearl shine agents may be used in the compositions as contemplated herein preferably in quantities from about 0.001 to about 5 wt %, more preferably from about 0.005 to about 4 wt %, particularly preferably from about 0.01 to about 3 wt % and most particularly from about 0.05 to about 2 wt % (for each) relative to the total weight thereof. Suitable pearl shine compositions and opacifiers are understood to include for example
Mono- and/or diesters of ethylene glycol, 1,2-Propanediol and/or glycerine having C8-C24 fatty acids,
Esters from polyethylene glycols having C8-C24 fatty acids,
TiO2 or TiO2-coated synthetic or natural mica and/or
Styrene/acrylate copolymers.

The opacifiers and/or pearl shine compositions known by the following INCI designators are particularly suitable: Glycol Distearates, such as the commercial product Cutina® AGS manufactured by Cognis, Glycol Monostearates, such as the commercial product Cutina® EGMS manufactured by Cognis, PEG-3 Distearates, such as the commercial product Genapol® TS manufactured by Clariant, PEG-2 Distearates, such as the commercial product Kessco® DEGMS manufactured by Akzo Nobel, Propylene Glycol Stearates, such as the commercial product Tegin® P manufactured by Goldschmidt and/or Styrene/Acrylate copolymers such as the commercial products Joncryl® 67 manufactured by Johnson Polymers, Suprawal® WS manufactured by BASF and/or Acusol® OP 301 manufactured by Rohm & Haas.

Further active ingredients, adjuvants and additives which may be included in the compositions as contemplated herein are for example:
Plant extracts,
Moisturisers,
Fragrances,
UV filters,
Thickeners such as gelatins or vegetable gums, for example Agar-Agar, Guar-Gum, Alginate, Xanthan-Gum, Gum arabicum, Karaya Gum, Locust Bean Gum, Linseed Gums, Dextrane, Cellulose derivatives, e.g. methyl cellulose, hydroxyalkyl cellulose and carboxymethyl cellulose, starch fractions and derivatives such as amylose, amylopectin and dextrin, clays and sheet silicates such as bentonite or fully synthetic hydrocolloids such as polyvinyl alcohol, the Ca-, Mg- or Zn-soaps,
Fibre structure improvement agents, particularly mono-, di- and oligosaccharides such as glucose, galactose, fructose, fruit sugar and lactose,
Dyes for coloring the composition,
Active ingredients such as bisabolol,
Ceramides. The group of ceramides is understood to include N-Acylsphingosine (fatty acidamides of sphingosine) or synthetic analogues of such lipids (also called “Pseudo-Ceramide”),
Propellants such as propane-butane mixtures, N2O, dimethylether, CO2 and air,
Antioxidants,
Preservatives, for example sodium benzoate or salicylic acid,
Viscosity adjusters such as salts (NaCl).

A second object as contemplated herein is a method for preserving the color of dyed keratinous fibers, in which a cosmetic compound comprising—relative to the weight thereof—

a) from about 0.01 to about 4.00 wt % of at least one alum and
b) from about 0.01 to about 5.00 wt % of at least one salt that contains a bivalent cation is applied to the keratinous fibers within a period of from about 5 seconds to about 24 hours before or after the hair is dyed and-or lightened.

A third object as contemplated herein is the use of at least one alum and at least one salt which contains a bivalent cation in cosmetic hair treatment compositions to protect the color of dyed hair. The notes regarding the compositions as contemplated herein apply mutatis mutandis for preferred embodiments of the method as contemplated herein and the use as contemplated herein.

### EXAMPLES

The following E compositions as contemplated herein were prepared (quantities are shown in wt %):

### a) Color Preserving Shampoos

| | Shampoo | Shampoo | Shampoo |
|---|---|---|---|
| | 1 | 2 | 3 |
| Sodium Laureth Sulphate | 11.00 | 11.00 | 10.00 |
| Cocamidopropyl Betaine | 1.00 | 1.50 | 3.00 |
| Disodium Cocoampho-diacetate | 0.50 | | |
| Cocamide MEA | 0.50 | 0.50 | |
| PEG-12 Dimethicone | 0.50 | 0.30 | |
| Glycol Distearate | 1.20 | | |
| PEG-7 Glyceryl Cocoate | 0.40 | 0.60 | 0.80 |
| Polyquaternium-10 | 0.90 | | 0.60 |
| Guar Hydroxypropyltrimonium | | 0.60 | |
| Chloride | | | |
| Panthenol | 0.30 | 0.20 | 0.20 |
| Dimethicone | 0.10 | | 0.50 |
| Hydrogenated Castor Oil | 0.20 | 0.10 | 0.30 |
| Calcium Lactate | 0.40 | 0.50 | 0.40 |
| KAl(SO4)2 × 12H2O | 0.80 | 1.00 | |
| NH4Al(SO4)2 × 12H2O | | | 0.75 |
| Preservative, fragrance, opt. acidifier | q.s. | q.s. | q.s. |
| Water | To 100 | To 100 | To 100 |
| pH | 4.5 | 4.4 | 4.5 |

After repeated washing (10-20 washes) with the shampoos described above, oxidatively dyed hair retains a vibrant color, of which the fastness properties are not significantly changed. The dyed hair also has a visually attractive luster and particularly soft texture.

### b) Conditioners

| | Conditioner | Conditioner | Conditioner |
|---|---|---|---|
| | 1 | 2 | 3 |
| Cetearyl alcohol | 2.50 | 2.50 | 3.00 |
| Quaternium-87 | 2.00 | 2.00 | 2.50 |
| Lactic acid | 1.60 | | |
| Propylene glycol | 0.50 | 0.50 | 1.00 |
| Isopropyl myristate | 0.30 | 0.30 | 0.50 |
| Distearoylethyl | 0.50 | 0.50 | 0.50 |
| hydroxyethylmonium | | | |
| methosulphate | | | |
| Calcium hydroxide | 0.54 | | |
| Phenoxyethanol | 0.40 | 0.40 | 0.60 |
| Stearamidopropyl | 0.30 | 0.30 | 0.40 |
| dimethylamine | | | |
| Fragrance | 0.25 | 0.25 | 0.25 |
| Sodium methylparabene | 0.20 | 0.20 | 0.20 |
| Polyquaternium-37 | 0.20 | 0.20 | 0.20 |
| Dicaprylyl carbonate | 0.20 | 0.20 | 0.20 |
| Panthenol | 0.10 | 0.10 | 0.10 |
| Benzophenone-4 | 0.05 | 0.05 | 0.05 |
| Amodimethicone/Morpholino- | 0.02 | 0.02 | 0.02 |
| methyl silsesquioxane | | | |
| copolymer | | | |
| Glycerin | 0.01 | 0.01 | 0.01 |
| Hydrolysed keratin | 0.01 | 0.01 | 0.01 |
| Calcium lactate | 0.80 | 1.40 | 1.40 |
| KAl(SO4)2 × 12H2O | 0.80 | 1.80 | |
| NH4Al(SO4)2 × 12H2O | | | 2.00 |
| Preservative, fragrance, opt. | q.s. | q.s. | q.s. |
| acidifier | | | |
| Water | To 100 | To 100 | To 100 |
| pH | 4.3 | 4.3 | 4.5 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Composition for treating keratinous fibers, comprising, relative to the weight thereof:
a) from about 0.01 to about 4.00 wt % of at least one alum; and
b) from about 0.01 to about 5.00 wt % of at least one salt which comprises a bivalent cation.

2. Composition according to claim 1, wherein the weight ratio between the at least one alum a) and the at least one salt b) is from about 4:1 to about 1:3.

3. Composition according to claim 1, having a pH value in the range from about 4.0 to about 5.0.

4. Composition according to claim 1, wherein the alum a) corresponds to the formula MIAl(SO₄)₂×12H₂O, in which MI stands for a potassium, sodium, ammonium or guanidinium ion.

5. Composition according to claim 1, comprising, relative to its weight, from about 0.05 to about 3.50 wt % of at least one alum.

6. Composition according to claim 1, wherein an organic or inorganic salt of copper, zinc, iron(II), calcium and/or magnesium is used as the salt b).

7. Composition according to claim 1, wherein calcium and/or magnesium lactate is used as the salt.

8. Composition according to claim 1, further comprising from about 0.05 to about 20.00 wt % of at least one surfactant, relative to its weight.

9. Composition according to claim 8, further comprising from about 1.00 to about 20.00 wt % of at least one anionic surfactant relative to its weight.

10. Composition according to claim 8, further comprising from about 0.10 to about 10.00 wt % of at least one cationic surfactant, relative to its weight.

11. Composition according to claim 1, further comprising from about 0.01 to about 3.00 wt % of at least one silicone, relative to its weight.

12. Composition according to claim 1, further comprising from about 0.01 to 3.00 wt % at least one cationic polymer, relative to its weight.

13. Composition according to claim 1, wherein the composition is configured to be applied before or after an oxidative treatment of keratinous fibers.

14. Method for protecting the color of dyed keratinous fibers, the method comprising applying a cosmetic compound to the keratinous fibers within a period of from about 5 seconds to about 24 hours before or after a dyeing and/or lightening procedure, the cosmetic compound comprising, relative to its weight,
a. from about 0.01 to about 4.00 wt % of at least one alum and
b. from about 0.01 to about 5.00 wt % of at least one salt which comprises a bivalent cation.

15. Composition according to claim 1, wherein the composition is utilized in cosmetic hair treatment compositions to preserve the color of dyed hair.

16. Composition according to claim 12, wherein the cationic polymer is a cationic polysaccharide polymer.

17. Composition according to claim 16, wherein the cationic polysaccharide polymer is a quaternized cellulose polymer.

18. Composition according to claim 5, wherein the alum is a potassium, sodium, or ammonium alum.

19. Composition according to claim 1, further comprising one or more protein hydrolysates in an amount of from about 0.01 to about 5 wt %, relative to the total weight thereof.

20. Composition for treating keratinous fibers, comprising, relative to the weight thereof:
a) from about 0.05 to about 3.50 wt % of at least one alum of formula MIAl(SO₄)₂×12H₂O, in which MI stands for a sodium ion, a potassium ion, an ammonium ion, or a guanidinium ion;
b) from about 0.02 to about 4.50 wt % of calcium lactate and/or magnesium lactate;
c) from about 2.00 to about 18.00 wt % of at least one anionic surfactant and/or from about 0.15 to about 8.00 wt % of at least one cationic surfactant; and
d) from about 0.01 to about 3.00 wt % of at least one of the cationic polymers known by the INCI designators Polyquaternium-10, Guar Hydroxypropyltrimonium Chloride, Polyquaternium-37, Polyquaternium-67 and/or Polyquaternium-72, and/or from about 0.01 to about 3.00 wt % of at least one silicone known by the INCI designator Dimethicone;
wherein the composition has a pH value in the range from about 4.0 to about 5.0.
